(19) United States
(12) Patent Application Publication
(10) Pub. No.: US 2017/0281573 A1
(43) Pub. Date: Oct. 5, 2017

(54) MEDICINAL COMPOSITION FOR ENHANCED DELIVERY OF TRITERPENES

(71) Applicants: PHARCON INC., Toronto (CA); RMW Pharma Consulting, Inc., Toronto (CA)

(72) Inventors: Joseph Schwarz, Toronto (CA); Michael Weisspapir, Toronto (CA)

(21) Appl. No.: 15/475,421

(22) Filed: Mar. 31, 2017

Related U.S. Application Data
(60) Provisional application No. 62/316,199, filed on Mar. 31, 2016.

(51) Int. Cl. A61K 31/19; A61K 9/107

(52) U.S. Cl. CPC A61K 31/19; A61K 9/1075

(57) ABSTRACT

The present disclosure relates to self-emulsifying oral compositions for enhanced absorption of poorly water soluble triterpenes, such as pentacyclic triterpene acids, either in substantially pure form or as a constituents of herbal extracts.

### CROSS REFERENCE TO RELATED APPLICATION

This application claims benefit of U.S. Provisional Patent Application No. 62/316,199 filed on Mar. 31, 2016, which is incorporated herein by reference in its entirety to the full extent permitted by law.

### BACKGROUND

Herbal extracts, containing triterpenes, have been used for alleviation of various diseases and health conditions for hundreds of years. Extracts of marjoram (Origanum majorana), Rosemary (Rosmarinus officinalis), Sage (Sage officinalis) herbs, Birch (Betula alba) leaves or bark, Olive tree (Olea europeae) leaves, Uva ursi (Arctostaphylos uva-ursi) leaves, Lavender (Lavandula angustifolia) leaves or flowers, apple (Malus domestica) peel, Loquat (Eriobotrya japonica) fruit and leaves, Banaba (Lagerstroemia speciosa) leaves, Olibanum (Boswellia sacra or B. serrata) leaves and bark and many others contain significant amounts of various triterpene acids, mainly pentacyclic triterpene acids (PTA) such as Ursolic, Betulinic, Oleanolic, Maslinic, Corosolic, Boswellic, Tormentic, Corosolic, Asiatic, Rosmarinic, Alphitolic, Glycyrrhetinic acid and other triterpenoids demonstrated efficacy in mitigation of heart diseases, obesity, blood pressure regulation, and can be used for treatment of cancer, skin disorders, inflammation, blood sugar control, weight control and other conditions. M. Broniatowski et al., “Interactions of Pentacyclic Triterpene Acids with Cardiolipins and Related Phosphatidylglycerols in Model Systems” Biochimica et Biophysica Acta (BBA)-Biomembranes 1838 (2014) 2530-2538.

Nevertheless, efficacy of the abovementioned extracts is seriously limited by low solubility of PTA compounds responsible for biological activity of these extracts, and by poor absorption associated with low solubility of highly hydrophobic triterpenes (log P>5).

Combining PTA with oils, such as rice bran oil, capric/caprylic glycerides, propylene glycol esters or castor oil can improve bioavailability compared with dry material. U.S. Pat. No. 7,713,546 describes a capsule containing 8-100 mg of Corosolic acid and rice bran oil and its application for weight-loss management and blood sugar balance. The soft gelatin capsule delivers an allegedly effective dose of Lagerstroemia speciosa L. and is marketed by Soft Gel Technologies under the trademark Glucosol™ for the assistance and maintenance of moderate weight loss through blood sugar maintenance. Based on the effects of corosolic acid on blood sugar levels, the product derives a healthy weight loss effect for Type II diabetics (non-insulin dependent) and healthy non-diabetics and improves absorption of an oil based delivery system.

Nevertheless, the increase of a triterpenoid component absorption remains insignificant compared to traditional dosage forms.

Incorporation of poorly soluble hydrophobic compounds into colloidal delivery formulations, such as self-emulsifying drug delivery systems (SEDDS) may noticeably improve absorption of these components. In order to increase drug bioavailability, U.S. Patent Publication No. 2008/0038335 teaches a composition formulation and method that alter the absorption site of orally administrated drugs and promote the absorption of bioactive lipophilic and/or hydrophilic compounds from the gastrointestinal tract. This formulation is administrated orally and comprises pharmaceuticals or nutraceuticals in combination with organic solvents and acid protectants. It increases the absorption in the stomach and avoids the undesired metabolism in the gastrointestinal tract thereby increasing their overall bioavailability.

SEDDS may efficiently improve bioavailability of incorporated hydrophobic compounds, but only if solubility of these compounds in the discontinuous (oil) phase of the emulsion, formed from the SEDDS after contact with water media, is high enough, or the component is highly potent and the required dose is low. Unfortunately, most of hydrophobic biologically active compounds have limited solubility in oily components traditionally used in SEDDS, thus restricting high loading of the delivery system.

Addition of polar organic solvents, such as alcohols, glycols, amides, ketones, sulfoxides, and pyrrolidones may improve drug solubility in the system. However, upon contact with water-containing media most of the poorly soluble active compounds precipitate immediately, thus decreasing absorption and bioavailability.

WO/2005/037250 discloses a self-emulsifying drug delivery system (SEDDS) comprising a hydrophilic surfactant, a digestible oil, and a non-aqueous protic solvent. It can be used in the administration of hydrophobic therapeutic compounds.

Powerful polar organic solvents, e.g., Dimethylsulfoxide (DMSO), Dimethylformamide (DMFA), Dimethylacetamide (DMAA), Ethylene glycol monoethyl ester (Transcutol), N-methylpyrrolidone (NMP) and other solvents may be irritating and toxic for live cells and cannot be given orally. Without the use of such solvents, achievable drug loading in SEDDS remains low, and usually does not exceed 2% w/w. U.S. Pat. No. 6,656,970; Xi, J. et al., “Formulation Development and Bioavailability Evaluation of a Self-Nanoemulsified Drug Delivery System of Oleanolic Acid” AAPS Pharm. Sci. Tech, (2009) Vol. 10, No. 1, pp. 172-182.

It was shown that the bioavailability of poorly soluble hydrophobic compounds administered orally as solution in digestible triglyceride oils may be significantly better than suspensions in water media. Inclusion of these drugs into lipid based colloidal delivery systems, such as emulsions, further improves drug absorption in the gastro-intestinal tract. Additionally, it was shown that emulsions with smaller droplets consistently provide better absorption compared with coarse emulsions. Diminishing of oil droplets from 4 mcm to 2 mcm increased cyclosporine absorption in small intestine approximately 1.7 folds. Tarr et al., “Enhanced intestinal absorption of Cyclosporine in rats through the reduction of emulsion droplet size” Pharm. Res. (1989) 6 (1) pp. 40-43. Nanoemulsions with droplets 30-150 nm are more efficient that emulsions with droplets 300-700 nm. Bioavailability of cyclosporine in SNEDDS (Sandimmune Neoral), forming fine oil-in-water nanoemulsion, is 174-239%, compared to bioavailability of SEDDS formulation (Sandimmune), forming coarse oil-in-water emulsion. De Shmidt et al., “Intestinal absorption of penclomedine from lipid vehicles in the conscious rat: contribution of emulsification versus digestibility” International Journal of Pharmaceutics 270 (2004) 109-118; Kang, B et al., “Development of self-microemulsifying drug delivery systems (SMEDDS) for oral bioavailability enhancement of simvastatin in beagle dogs” International Journal of Pharmaceutics 274 (2004) 65-73; Mueller E. et al., “Influence of a fat-rich meal on the pharmacokinetics of a new oral formulation of cyclosporine in a crossover comparison with the market formulation” Pharm. Res. (1994) 11 (1) pp. 151-5; “Improved dose linearity of cyclosporine pharmacokinetics from a microemulsion formulation. Pharm. Res. (1994) 11(2):301-4; Myers R. et al., “Systemic bioavailability of penclomedine (NSC-338720) from oil-in-water emulsions administered intraduodenally to rats” International Journal of Pharmaceutics, 78 (1992) pp. 217-226.

Terpenoids, including pentacyclic triterpene acids, can also be incorporated into SEDDS in order to improve oral absorption, as described in abovementioned U.S. Patent Publication No. 2008/0038335. Nonetheless, the described self-nanoemulsifying formulations, based on triglycerides or glycol esters, do not possess required solubilisation capacities. Solubility of PTAs in these vehicles usually does not exceed 2-4% by weight and does not allow to obtain solid dosage forms with high drug loading.

The use of polar water miscible organic solvents may improve solubilisation of PTAs in SEDDS, but after contact with water media most of the active compound precipitates almost immediately.

Another drawback of such SEDDS is the formation of relatively large oil droplets when composition containing high amount of polar solvent is mixed with water phase. To obtain small droplet size of the formed emulsions an extremely high concentration of surfactant should be used (usually 30-50%), which is associated with potential cell toxicity. Buyukorturk F., et al., “Impact of emulsion-based drug delivery systems on intestinal permeability and drug release kinetics”. Journal of Controlled Release 142 (2010) pp. 22-30.

There is still an unmet need for a safe and effective delivery system for hydrophobic triterpene acids with high load capacity and ability to overcome the described limitations. Such self-emulsifying composition should easily form an oil in water emulsion with small droplets and provide high bioavailability of incorporated biologically active terpene acid. The oil phase of such SEDDS should keep biologically active components in mostly dissolved stage, providing effective absorption of the colloidal particles with incorporated active components.

### SUMMARY

The present disclosure relates to self-emulsifying oral compositions for enhanced absorption of triterpenes, such as pentacyclic triterpene acids, either in pure form or as a constituents of herbal extracts.

In one embodiment, the proposed composition is a self-emulsifying concentrate, spontaneously forming an oil-in-water emulsion upon contact with water media, such as drinking water, fruit juice, gastric fluid, intestinal fluid or saliva.

In another embodiment, the biologically active components of the composition are hydrophobic compounds, poorly soluble in water, and substantially dissolved in the self-emulsifying composition.

In yet another embodiment, biologically active hydrophobic compounds remain substantially dissolved in the oil phase after formation of the oil-in-water emulsion.

In one embodiment, the composition contains a solubilizer to dissolve the biologically active hydrophobic components. The solubilizer can be a liquid or a solid aromatic compound, selected from the group of physiologically acceptable chromones, chromanes, tocols, tocopherols and tocopherol esters, tocotrienoles, benzoic acid esters, hydroxybenzoic acid esters, eugenol, anisole, anethole, flavones, isoflavones, flavonoids, indoles, quinones, ubidecarenone, curcuminoids, derivatives and mixtures thereof.

In another embodiment, the self-emulsifying composition comprises alpha-lipoic acid in form of free R-(+)-alpha-lipoic acid, S-(−)-alpha-lipoic acid, racemic alpha-lipoic acid or physiologically acceptable salt thereof.

In yet another embodiment, incorporation of alpha-lipoic acid causes a decrease of the droplet size of the formed oil-in-water emulsion loaded with triterpene acid.

In another embodiment, the oil droplets of the spontaneously formed emulsion have average size smaller than about 1000 nm, preferably less than about 500 nm, more preferably smaller than about 300 nm, most preferably smaller than about 150 nm.

In another embodiment, the composition contains natural or synthetic phospholipid as an co-emulsifier, selected from group of soy or egg lecithins, phophatidic acids, phosphatidylcholines, phosphatidylserines, phosphatidylethanolamines, hydrogenated lecithins, di stearoylphosphatidylcholine, dioleoylphosphatidylcholine, analogs and mixtures thereof.

In one embodiment, the composition may additionally contain a lipid component selected from group of medium and long chain mono-, di- and triglycerides, fatty acid esters of propylene glycol, propylene glycol caprylates and laurates, macrogol oleyl- and linoleyl glycerides (Labrafil M1944CS and Labrafil M2125CS).

In another embodiment, the composition can additionally contain physiologically acceptable additives, antioxidants, sweeteners, flavors, colorants, preservatives, taste-masking components and UV protectants.

In another embodiment, the proposed formulation can be administered orally in liquid filled hard shell capsules or in softgels, in powder forms after mixing with appropriate absorbents, compressed into the tablets, in liquid form (“as is”) as a self-emulsifying concentrate or as an emulsion after dilution with water media.

Other embodiments, objects, features, and advantages will be set forth in the detailed description of the embodiments that follow and, in part, will be apparent from the description or may be learned by practice of the claimed disclosure. These objects and advantages will be realized and attained by the compositions and methods described and claimed herein. The foregoing Summary has been made with the understanding that it is to be considered as a brief and general synopsis of some of the embodiments disclosed herein, is provided solely for the benefit and convenience of the reader, and is not intended to limit in any manner the scope, or range of equivalents, to which the appended claims are lawfully entitled. It should be recognized that the embodiments above may additionally comprise fillers, glidants, flavoring agents, lubricants, and preservatives.

### DETAILED DESCRIPTION

While the present disclosure is capable of being embodied in various forms, the description below of several embodiments is made with the understanding that the present disclosure is to be considered as an exemplification of the claimed subject matter, and is not intended to limit the appended claims to the specific embodiments illustrated and/or described, and should not be construed to limit the scope or breadth of the present disclosure. The headings used throughout this disclosure are provided for convenience only and are not to be construed to limit the claims in any way. Embodiments illustrated under any heading may be combined with embodiments illustrated under any other heading.

Herbal extracts containing PTA (e.g., Loquat/Eryobotrya or Banaba extracts, containing Corosolic acid; Rosemary extract containing Ursolic acid; Boswellia serrata extract containing Boswellic acid; and many others) demonstrate different biological activities and may be used for alleviation of various pathological conditions such as elevated blood sugar and high cholesterol levels, for treatment of systemic inflammation, improvement of skin health and many other indications. A serious limitation for effective use of PTA-containing extracts is the extremely low water solubility of either PTA or salts thereof. For example, water solubility of Corosolic acid is below 1 mcg/ml, and solubility of sodium salt of this acid is less than 5 mcg/ml. Other PTAs demonstrate similar solubility. Due to extremely restricted solubility, absorption of PTA in the body is low, and biological activity is seriously limited for oral administration route. Additionally, despite high lipophilicity, most PTAs have low solubility in fixed oils due to polycyclic structure of the molecules.

Surprisingly, it was found that incorporation of some aromatic compounds into the oil phase of the self-emulsifying composition may seriously improve solubilization of PTA in the mixture of the oil and aromatic component. Such effect was observed for ubiquinone, tocotrienols, tocopherols and tocopherol esters, curcumins, aromatic esters and some other compounds. The solubility has increased not only for liquid compounds, such as aromatic esters or ethers (e.g., esters of hydroxybenzoic acid, anethole, anisole or eugenol) but also for solid components such as ubiquinone or curcumin.

The incorporation of aromatic molecules into self-emulsifying formulations usually leads to the formation of coarse emulsions compared with traditional formulations, based on glycerides or propylene glycol esters. Absorption of biologically active hydrophobic components from coarse emulsions is usually lower than from submicron colloidal systems.

It was unexpectedly found that addition of alpha-lipoic acid (“ALA”) to SEDDS causes a noticeable decrease of droplet size of the formed emulsions. This effect is not associated with a surfactant activity of alpha-lipoic acid since other carboxylic acids with similar properties, e.g., oleic, capric or caprylic, do not diminish droplet size of the emulsions. The addition of ALA in physiologically reasonable amounts (10-300 mg per dose) leads to increase of the submicron population of the formed emulsions, and for most cases results in a nanoemulsion with narrow size distribution.

### I. DEFINITIONS

For convenience, before further description of the present teachings, certain terms employed in the specification, examples, and appended claims are collected here. These definitions should be read in light of the remainder of the disclosure and as understood by a person of ordinary skill in the art. Unless defined otherwise, all technical and scientific terms used herein have the same meaning as commonly understood by a person of ordinary skill in the art.

A. General Terms

The use of the terms “a,” “an” and “the” and similar references in the context of this disclosure (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., such as, preferred, preferably) provided herein, is intended merely to further illustrate the content of the disclosure and does not pose a limitation on the scope of the claims. No language in the specification should be construed as indicating any non-claimed element as essential to the practice of the present disclosure.

The phrase “and/or,” as used herein, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements).

As used herein, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of” “only one of” or “exactly one of” “Consisting essentially of”, when used in the claims, shall have its ordinary meaning as used in the field of patent law.

As used herein, the phrase “at least one” in reference to a list of one or more elements should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, all transitional phrases such as “comprising,” “including,” “carrying,” “having,” “containing,” “involving,” “holding,” “associated,” “associated with,” and the like are to be understood to be open-ended, i.e. to mean including but not limited to.

The use of individual numerical values are stated as approximations as though the values were preceded by the word “about” or “approximately.” Similarly, the numerical values in the various ranges specified in this application, unless expressly indicated otherwise, are stated as approximations as though the minimum and maximum values within the stated ranges were both preceded by the word “about” or “approximately.” In this manner, variations above and below the stated ranges can be used to achieve substantially the same results as values within the ranges. As used herein, the terms “about” and “approximately” when referring to a numerical value shall have their plain and ordinary meanings to a person of ordinary skill in the art to which the disclosed subject matter is most closely related or the art relevant to the range or element at issue. The amount of broadening from the strict numerical boundary depends upon many factors. For example, some of the factors which may be considered include the criticality of the element and/or the effect a given amount of variation will have on the performance of the claimed subject matter, as well as other considerations known to those of skill in the art. As used herein, the use of differing amounts of significant digits for different numerical values is not meant to limit how the use of the words “about” or “approximately” will serve to broaden a particular numerical value or range. Thus, as a general matter, “about” or “approximately” broaden the numerical value. Also, the disclosure of ranges is intended as a continuous range including every value between the minimum and maximum values plus the broadening of the range afforded by the use of the term “about” or “approximately.” Thus, recitation of ranges of values herein are merely intended to serve as a shorthand method of referring individually to each separate value falling within the range, unless otherwise indicated herein, and each separate value is incorporated into the specification as if it were individually recited herein.

The phrase “substantially pure” refers to a substance having total purity of greater than 90%, specifically greater than 95%, more specifically greater than 98%, and most specifically greater than 99%. For example, the phrase “substantially pure substance A” means substance A is at least 90% pure with respect to all impurities, specifically substance A is at least 95% pure with respect to all impurities, more specifically substance A is at least 98% pure with respect to all impurities, and most specifically substance A is at least 99% pure with respect to all impurities.

A “subject” or a “patient” refers to any mammal (e.g., a human), such as a mammal that may be susceptible to a disease or disorder. Examples include a human, a non-human primate,a cow, a horse, a pig, a sheep, a goat, a dog, a cat, or a rodent such as a mouse, a rat, a hamster, or a guinea pig. In various embodiments, a subject refers to one that has been or will be the object of treatment, observation, or experiment.

B. Terms Related to Compositions of the Present Disclosure

“Lipid” refers to a fatty or waxy organic compound that is readily soluble in nonpolar solvent (e.g. ether) but not in polar solvent (e.g water). Its major biological functions involve energy storage, structural component of cell membrane, and cell signaling. Examples of lipids are waxes, monoglycerides, diglycerides, triglycerides (edible oils, fats), fat-soluble vitamins, sterols, cholesterol, and phospholipids.

A “surfactant” refers to an organic compound that contains both a hydrophobic group and a hydrophilic group. The hydrophilic group is often referred to as the head and the hydrophobic group as the tail. A surfactant will adsorb at interfaces between hydrophilic compositions, such as oil, and hydrophilic compositions, such as water, wherein the hydrophilic head will extend into the water and the hydrophobic tail will extend into the oil.

As used herein, “oral administration” refers to treatment of a disease or disorder by delivery of therapeutically effective agents through the mouth. The agent may permeate through the oral mucosa or anywhere throughout the gastrointestinal tract. Oral administration includes, but is not limited to, solid dosage forms such as tablet, chewable tablet, lozenge, powder, dissolving film, gum, as well as homogenous and heterogeneous liquids, including emulsions.

As used herein, “treat,” “treatment” or “treating” refers to an amelioration of a disease or disorder, or at least one sign or symptom thereof. In another embodiment, “treatment” or “treating” refers to an amelioration of at least one measurable physical parameter, not necessarily discernible by the patient. In yet another embodiment, “treatment” or “treating” refers to reducing the progression of a disease or disorder, for example, by reducing the rate of disease progression compared to a reference population having the same disease or decreasing the degree or rate or progression of a sign or symptom in the subject prior to treatment. In yet another embodiment, “treatment” or “treating” refers to delaying the onset of a disease or disorder, e.g., compared to a reference population or other method of determining such a parameter as is known by those in the art.

The phrase “therapeutically effective amount” as used herein means that amount of therapeutic effective agent that is effective for producing a desired therapeutic effect. Accordingly, a therapeutically effective amount treats or prevents a disease or a disorder, ameliorates at least one sign or symptom of the disorder, e.g., lowers a diabetic patient's glucose level.

The term “therapeutic effect” is art-recognized and refers to a local or systemic effect in animals, particularly mammals, and more particularly humans caused by a pharmacologically active substance. The term thus means any substance intended for use in the diagnosis, cure, mitigation, treatment or prevention of disease or in the enhancement of desirable physical or mental development and conditions in an animal or human.

The term “bioavailable” is art-recognized and refers to a form of the subject disclosure that allows for it, or a portion of the amount administered, to be absorbed by, incorporated to, or otherwise physiologically available to a subject or patient to whom it is administered.

### II. THE COMPOSITION

In various embodiments, herbal extracts include those of marjoram (Origanum majorana), Rosemary (Rosmarinus officinalis), Sage (Sage officinalis) herbs, Birch (Betula alba) leaves or bark, Olive tree (Olea europeae) leaves, Uva ursi (Arctostaphylos uva-ursi) leaves, Lavender (Lavandula angustifolia) leaves or flowers, apple (Malus domestica) peel, Loquat (Eriobotrya japonica) fruit and leaves, Banaba (Lagerstroemia speciosa) leaves, Olibanum (Boswellia sacra or B. serrata) leaves and bark and many others.

In various embodiments, the plant extract is present in an amount between about 0.10% and about 90%, between about 0.2% and about 85%, between about 0.5% and about 80%, between about 1.0% and 75%, between about 10% and about 70%, between about 20% and about 60%, between about 30% and about 50%, between about 40% and about 45% by weight of the composition. In other embodiments, the plant extract is about 0.10%, 0.50%, 0.75%, 0.10%, 1.25%, 1.50%, 1.75%, 2.0%, 2.25%, 2.50%, 2.75%, 3.00%, 3.25%, 3.50%, 3.75%, 4.00%, 4.25%, 4.50%, 4.75%, 5.0%, 5.25%, 5.50%, 5.75%, 6.00%, 6.25%, 6.50%, 6.75%, 7.00%, 7.25%, 7.50, 7.75%, 8.00%, 8.25%, 8.50%, 8.75%, 9.00%, 9.25%, 10.00%, 11.00%, 12.00%, 13.00%, 14.00%, 15.00%, 16.00%, 17.00%, 18.00%, 19.00%, 20.00%, 22.50%, 25.00%, 27.50%, 30%, 35%, 40%, 50%, 60%, 70%, 80%, or 90% by weight of the composition.

In one embodiment, the plant extract is present in an amount from about 0.01% to about 80% by weight of composition.

In various embodiments, the triterpene acid is present in an amount between about 0.10% and about 90%, between about 0.2% and about 85%, between about 0.5% and about 80%, between about 1.0% and 75%, between about 10% and about 70%, between about 20% and about 60%, between about 30% and about 50%, between about 40% and about 45% by weight of the composition. In other embodiments, the triterpene acid is about 0.10%, 0.50%, 0.75%, 0.10%, 1.25%, 1.50%, 1.75%, 2.0%, 2.25%, 2.50%, 2.75%, 3.00%, 3.25%, 3.50%, 3.75%, 4.00%, 4.25%, 4.50%, 4.75%, 5.0%, 5.25%, 5.50%, 5.75%, 6.00%, 6.25%, 6.50%, 6.75%, 7.00%, 7.25%, 7.50, 7.75%, 8.00%, 8.25%, 8.50%, 8.75%, 9.00%, 9.25%, 10.00%, 11.00%, 12.00%, 13.00%, 14.00%, 15.00%, 16.00%, 17.00%, 18.00%, 19.00%, 20.00%, 22.50%, 25.00%, 27.50%, 30%, 35%, 40%, 50%, 60%, 70%, 80%, or 90% by weight of the composition.

In one embodiment, the triterpene acid is present in an amount from about 0.01% to about 80% by weight of composition.

In one embodiment, the proposed composition is a self-emulsifying concentrate, spontaneously forming an oil-in-water emulsion upon contact with water media, such as drinking water, fruit juice, gastric fluid, intestinal fluid or saliva.

In yet another embodiment, biologically active hydrophobic compounds remain substantially dissolved in the oil phase after formation of the oil-in-water emulsion.

In still other embodiments, the self-nanoemulsifying composition forms a submicron emulsion, nanoemulsion, microemulsion, picoemulsion or micellar solution.

As used herein, an “oil” may be a solid or a liquid at 23° C., but when in a liquid state the oil is immiscible with water. As used herein, “immiscible” refers to compounds that fail to mix in all proportion to form a homogenous solution. When an oil and water are mixed, the two substances may separate to form two separate homogenous layers, form a plurality of oil-in-water particles, or a combination thereof. In certain embodiments, the oil is amphiphilic, i.e., having a hydrophilic portion and a hydrophobic portion, or a relatively hydrophilic portion and a relatively hydrophobic portion.

In various embodiments a composition comprises one or more surfactants. In some embodiments the surfactant is between about 0.10% and about 90%, between about 0.2% and about 85%, between about 0.5% and about 80%, between about 1.0% and 75%, between about 10% and about 70%, between about 20% and about 60%, between about 30% and about 50%, between about 40% and about 45% by weight of the composition. In other embodiments, the surfactant is about 0.10%, 0.50%, 0.75%, 0.10%, 1.25%, 1.50%, 1.75%, 2.0%, 2.25%, 2.50%, 2.75%, 3.00%, 3.25%, 3.50%, 3.75%, 4.00%, 4.25%, 4.50%, 4.75%, 5.0%, 5.25%, 5.50%, 5.75%, 6.00%, 6.25%, 6.50%, 6.75%, 7.00%, 7.25%, 7.50, 7.75%, 8.00%, 8.25%, 8.50%, 8.75%, 9.00%, 9.25%, 10.00%, 11.00%, 12.00%, 13.00%, 14.00%, 15.00%, 16.00%, 17.00%, 18.00%, 19.00%, 20.00%, 22.50%, 25.00%, 27.50%, 30%, 35%, 40%, 50%, 60%, 70%, 80%, or 90% by weight of the composition.

In various embodiments, the solubilizer is present in an amount between about 0.10% and about 90%, between about 0.2% and about 85%, between about 0.5% and about 80%, between about 1.0% and 75%, between about 10% and about 70%, between about 20% and about 60%, between about 30% and about 50%, between about 40% and about 45% by weight of the composition. In other embodiments, the plant extract is about 0.10%, 0.50%, 0.75%, 0.10%, 1.25%, 1.50%, 1.75%, 2.0%, 2.25%, 2.50%, 2.75%, 3.00%, 3.25%, 3.50%, 3.75%, 4.00%, 4.25%, 4.50%, 4.75%, 5.0%, 5.25%, 5.50%, 5.75%, 6.00%, 6.25%, 6.50%, 6.75%, 7.00%, 7.25%, 7.50, 7.75%, 8.00%, 8.25%, 8.50%, 8.75%, 9.00%, 9.25%, 10.00%, 11.00%, 12.00%, 13.00%, 14.00%, 15.00%, 16.00%, 17.00%, 18.00%, 19.00%, 20.00%, 22.50%, 25.00%, 27.50%, 30%, 35%, 40%, 50%, 60%, 70%, 80%, or 90% by weight of the composition.

In one embodiment, the solubilizer is present in an amount from about 0.5% to about 80% by weight of composition.

In various embodiments, self-emulsifying composition forms oil droplets with an average size of less than about 500 nm, 400 nm, 300 nm, 250 nm, 200 nm, 180 nm, 150 nm, 120 nm, 100 nm, 90 nm, 80 nm, 70 nm, 60 nm, 50 nm, 40 nm, 30 nm or 20 nm. In other embodiments, the oil droplets has an average size of 10 nm, 20 nm, 30 nm, 40 nm, 50 nm, 60 nm, 70 nm, 80 nm, 90 nm, 100 nm, 120 nm, 150 nm, 180 nm, 200 nm, 250 nm or 300 nm. In further embodiments, the oil droplets has an average size of 10-500 nm, 10-400 nm, 10-300 nm, 10-250 nm, 10-200 nm, 10-150 nm, 10-100 nm, 10-75 nm, 10-50 nm, 50-500 nm, 50-400 nm, 50-300 nm, 50-200 nm, 50-150 nm, 50-100 nm, 50-75 nm, 100-500 nm, 100-400 nm, 100-300 nm, 100-250 nm, 100-200 nm, 100-150 nm, 150-500 nm, 150-400 nm, 150-300 nm, 150-250 nm, 150-200 nm, 200-500 nm, 200-400 nm, 200-300 nm, 200-250 nm, 200-500 nm, 200-400 nm or 200-300 nm.

In another embodiment, the oil droplets of the spontaneously formed emulsion have average size smaller than about 1000 nm, preferably less than about 500 nm, more preferably smaller than about 300 nm, most preferably smaller than about 150 nm.

In one embodiment, the solubilizer can be a liquid or a solid aromatic compound, selected from the group of physiologically acceptable chromones, chromanes, tocols, tocopherols and tocopherol esters, tocotrienoles, benzoic acid esters, hydroxybenzoic acid esters, eugenol, anisole, anethole, flavones, isoflavones, flavonoids, indoles, quinones, ubidecarenone, curcuminoids, derivatives and mixture thereof.

In another embodiment, the self-emulsifying composition comprises alpha-lipoic acid in form of free R-(+)-alpha-lipoic acid, S-(−)-alpha-lipoic acid, racemic alpha-lipoic acid or physiologically acceptable salt thereof.

In various embodiments, the R-alpha-lipoic acid, S-alpha-lipoic acid, racemic alpha-lipoic acid or a physiologically acceptable salt thereof is present in an amount between about 0.10% and about 90%, between about 0.2% and about 85%, between about 0.5% and about 80%, between about 1.0% and 75%, between about 10% and about 70%, between about 20% and about 60%, between about 30% and about 50%, between about 40% and about 45% by weight of the composition.

In one embodiment, the R-alpha-lipoic acid, S-alpha-lipoic acid, racemic alpha-lipoic acid or a physiologically acceptable salt thereof is present in an amount from about 0.5% to about 50% by weight of the composition.

In yet another embodiment, incorporation of alpha-lipoic acid causes decrease of the droplet size of the formed oil-in-water emulsion, loaded with triterpene acid.

In another embodiment, the composition contains natural or synthetic phospholipid as an co-emulsifier. In various embodiments, the phospholipid is selected from group consisting of soy or egg lecithins, phophatidic acids, phosphatidylcholines, phosphatidylserines, phosphatidylethanolamines, hydrogenated lecithins, di stearoylphosphatidylcholine, dioleoylphosphatidylcholine, analogs and mixtures thereof.

In one embodiment, the composition may additionally contain a lipid component selected from group of medium and long chain mono-, di- and triglycerides, fatty acid esters of propylene glycol, propylene glycol caprylates and laurates, macrogol oleyl- and linoleyl glycerides (Labrafil M1944CS and Labrafil M2125CS).

In various embodiments, the phospholipid is present in an amount between about 0.01% and about 90%, between about 0.05% and about 85%, between about 0.1% and about 80%, between about 0.1% and 70%, between about 0.1% and about 60%, between about 0.1% and about 65%, between about 0.1% and about 50% by weight of the composition. In other embodiments, the phospholipid is about 0.10%, 0.50%, 0.75%, 0.10%, 1.25%, 1.50%, 1.75%, 2.0%, 2.25%, 2.50%, 2.75%, 3.00%, 3.25%, 3.50%, 3.75%, 4.00%, 4.25%, 4.50%, 4.75%, 5.0%, 5.25%, 5.50%, 5.75%, 6.00%, 6.25%, 6.50%, 6.75%, 7.00%, 7.25%, 7.50, 7.75%, 8.00%, 8.25%, 8.50%, 8.75%, 9.00%, 9.25%, 10.00%, 11.00%, 12.00%, 13.00%, 14.00%, 15.00%, 16.00%, 17.00%, 18.00%, 19.00%, 20.00%, 22.50%, 25.00%, 27.50%, 30%, 35%, 40%, 50%, 60%, 70%, 80%, or 90% by weight of the composition.

In one embodiment, the phospholipid is present in an amount from about 0.1% to about 50% by weight of composition.

In another embodiment, the composition can additionally contain physiologically acceptable additives, antioxidants, sweeteners, flavors, colorants, preservatives, taste-masking components and UV protectants.

In another embodiment, the proposed formulation can be administered orally in liquid filled hard shell capsules or in softgels, in powder forms after mixing with appropriate absorbents, compressed into the tablets, in liquid form (“as is”) as a self-emulsifying concentrate or as an emulsion after dilution with water media.

### III. EXAMPLES

The following examples illustrate the features and scope of the present invention. These examples should not be considered as any limitations, but should be merely interpreted to teach how to prepare a medical compositions that provide an enhanced delivery system of Triterpenes.

### Example 1

### Tablet Preparation

Tablets with incorporated PTA-containing herbal extract and with or without alpha-lipoic acid were prepared by traditional wet granulation technique, Table 1. Active components after combining and mixing with fillers, polyols, sweeteners, acidity regulator and other excipients were granulated with solution of polymeric binder (Polyvinylpyrrolidone PVP K-25) or Hydroxypropylcellulose (Klucel MXF) in ethanol. After drying, the granulation was screened through screen (18 mesh) and mixed with glidant and lubricant. Tablets were compressed using an appropriate tablet press.

### Example 2

### Capsule Preparation

Composition for filling capsules was obtained by mixing of all ingredients until a homogenous formulation was obtained, Table 2 and Table 3. To accelerate the process, the system could be heated correspondently under nitrogen or other inert gas atmosphere. For filling of hard-shell capsules a semi-solid consistency is preferable. Soft-shell capsules could be filled with liquid compositions.

TABLE 1
Tablets containing self-emulsifying composition with herbal extract containing Corosolic acid.

| | Ex. | Ex. | Ex. | Ex. | Ex. | Ex. | Ex. | Ex. | Ex. | Ex. | Ex. | Ex. |
|---|---|---|---|---|---|---|---|---|---|---|---|---|
| | 1 | 2 | 3 | 4 | 5 | 6 | 7 | 8 | 9 | 10 | 11 | 12 |
| COMPONENT | Per tablet, mg | | | | | | | | | | | |
| Banaba leaves extract | 1 | 3 | 3 | 3 | 3 | 3 | 3 | 3 | 3 | 6 | 6 | 6 |
| (18% corosolic acid) | | | | | | | | | | | | |
| Rac-alpha-Lipoic acid | | | | | | | | | 30 | 30 | 50 | 100 |
| Tocopherol acetate | 3 | 2 | 2 | 3 | 3 | 3 | 10 | 15 | 25 | 28 | 8 | 8 |
| Tocopherol | | | | | | | | | | 22 | 22 | 22 |
| Peppermint oil | | 2 | 2 | 2 | 2 | 2 | 4 | | | 10 | 11 | 11 |
| Anethole | | | | | | | | 5 | 5 | | | |
| Lecithin | 2 | 4 | 2 | 1 | 1 | 2 | 2 | 2 | 2 | 2 | | |
| PEG 40 stearate | 15 | 15 | 15 | 15 | 15 | 15 | 15 | 15 | 15 | 25 | 24 | 24 |
| PVP K 25 | 5 | | | | | | | | | | | |
| Microcryst. cellulose | 60 | 30 | 30 | 40 | 40 | 40 | 40 | 50 | 50 | 60 | 60 | 60 |
| Silicon dioxide | 5 | 5 | 5 | 5 | 50 | 20 | 30 | 30 | 70 | 50 | 50 | 50 |
| Ethanol for granulation | q.s. | q.s. | q.s. | q.s. | q.s. | q.s. | q.s. | q.s. | q.s. | q.s. | q.s. | q.s. |
| Hydroxypropylcellulose | | 10 | 20 | 18 | 18 | 18 | 15 | 12 | 12 | 18 | 24 | 6 |
| Alginic acid | 15 | 15 | 15 | 15 | | 15 | | 15 | 25 | 15 | 15 | 15 |
| Mannitol | | 100 | | 100 | 100 | 100 | 150 | 150 | 150 | 250 | 250 | 250 |
| Xylitol | 100 | 100 | 100 | 100 | 100 | | 150 | | 150 | | | |
| Sorbitol | | | | | | 100 | | 100 | | 150 | 150 | 150 |
| Isomalt | | | 150 | | | | | | | | | |
| Dibasic calcium phosphate | | | | 100 | 60 | 50 | 60 | 30 | 30 | 50 | 50 | 50 |
| (anhydrous) | | | | | | | | | | | | |
| Flavor | | | | | | | | | 5 | 16 | 12 | 20 |
| Tartaric acid | | 2 | 5 | 10 | 10 | 10 | 15 | 15 | 15 | 15 | | |
| Sucralose | 3 | 2 | 2 | 2 | 2 | 2 | 2 | 2 | 5 | 5 | | |
| Crosspovidone | 10 | | | | | | | | | | | |
| Pregelatinized starch | | 10 | 10 | | | | | | | | | |
| Maltodextrin | | | | | 90 | 50 | 60 | 40 | 40 | 60 | 60 | 60 |
| Stearic acid | 2 | 2 | 2 | 2 | 5 | | | | | | | |
| Glycine | | | | | | | | | 25 | | | |
| PEG 3350 | | | | | | 20 | | 25 | 25 | 30 | 35 | 35 |
| Total weight, mg | 261 | 302 | 363 | 416 | 499 | 450 | 556 | 509 | 679 | 839 | 827 | 867 |

TABLE 2
Capsules containing self-emulsifying composition with single extract

| | Ex. | Ex. | Ex. | Ex. | Ex. | Ex. | Ex. | Ex. | Ex. | Ex. | Ex. | Ex. |
|---|---|---|---|---|---|---|---|---|---|---|---|---|
| | 13 | 14 | 15 | 16 | 17 | 18 | 19 | 20 | 21 | 22 | 23 | 24 |
| COMPONENT | Per capsule, mg | | | | | | | | | | | |
| Banaba leaves extract | 2 | 6 | 6 | 6 | 6 | 6 | 6 | 9 | 9 | 6 | 12 | 9 |
| (18% corosolic acid) | | | | | | | | | | | | |
| R-Alpha-Lipoic acid (Na salt) | | | 12 | | | | | | | | | |
| rac-Alpha-Lipoic acid | 100 | 100 | | 75 | 115 | 100 | 75 | 80 | 120 | 75 | 100 | 200 |
| MCT oil (capric/caprylic | 160 | 180 | 300 | 300 | 300 | 100 | 360 | 240 | 280 | | 240 | |
| triglycerides) | | | | | | | | | | | | |
| Capric/caprylic mono and | 240 | 200 | | | | 150 | | | | | | 190 |
| diglycerides (Capmul MCM) | | | | | | | | | | | | |
| Acetylated mono- and diglycerides | | | | | | | | | | 300 | | |
| Oleoyl polyoxyl-6 glycerides | | | | | | | | | | | 120 | 150 |
| (Labrafil M1944S) | | | | | | | | | | | | |
| Caprylocaproyl macrogol-8 | | | | | | | | 120 | | | | 90 |
| glycerides (Labrasol) | | | | | | | | | | | | |
| D-Alpha-Tocopherol acetate | 80 | 60 | 75 | 75 | 75 | 50 | | 40 | | | 55 | 30 |
| DL-alpha Tocopherol | | | | | | 30 | 45 | | | 48 | | 20 |
| Gamma-Tocopherol | | | | | | | 25 | | 60 | | | |
| Tocotrienols (natural mixture) | | | | | | | | 30 | | 12 | | |
| Anethole | | | | | | | | | 10 | 10 | | |
| Curcumin | | | | | | 30 | | | | | | |
| Gaultheria procumbens essential oil | | | | | | | | 20 | | | | |
| Ubidecarenone | | | | | | 30 | | | 50 | | | |
| Peppermint essential oil | | | | | | | | 10 | 30 | | | 10 |
| Anise oil | | | | | | | 10 | | | | | |
| Clove oil | | | | | | | 5 | | | | | |
| Lecithin | 45 | 45 | 45 | 45 | 45 | 50 | 45 | 30 | 50 | 45 | 45 | 45 |
| Polysorbate 60 | 100 | 150 | 120 | 120 | 120 | | | | | | | |
| Polysorbate 20 | | | | | | | | | | | 162 | |
| Polysorbate 80 | | | | | | 120 | 150 | | | | | |
| Hydrogenated polyethoxylated | | | | | | | | | 100 | 136 | | |
| castor oil | | | | | | | | | | | | |
| Polyethoxylated castor oil | 60 | | | | | | | | | | | 114 |
| PEG-15 Hydroxystearate | | | | | | | | 120 | | | | |
| Sorbitan monooleate (Span 80) | | 55 | 44 | | 50 | | 54 | | | | | 86 |
| PEG 40 stearate | | | | 60 | | | | | | 66 | | |
| Tocophersolan (TPGS) | | | | | | | | 60 | 50 | | | |
| Tyloxapol | | | | | | 60 | | | | | 58 | |
| Total weight, mg | 787 | 796 | 602 | 681 | 711 | 806 | 775 | 759 | 759 | 698 | 792 | 944 |

TABLE 3
Capsules containing self-emulsifying compositions with pure PTA or with multiple extracts

| | Ex. | Ex. | Ex. | Ex. | Ex. | Ex. | Ex. | Ex. | Ex. | Ex. | Ex. | Ex. |
|---|---|---|---|---|---|---|---|---|---|---|---|---|
| | 25 | 26 | 27 | 28 | 29 | 30 | 31 | 32 | 33 | 34 | 35 | 36 |
| COMPONENT | Per capsule, mg | | | | | | | | | | | |
| Olive fruit extract (25% Maslinic acid) | | | 12 | | | | | | | | | 30 |
| Ursolic acid (purity >95%) | 50 | | | | | | | 100 | | | | |
| Glycyrrhetinic acid (Enoxolone) | | | 30 | | | | | | | 50 | | 20 |
| Milk thistle extract | | | | | | | | 40 | 80 | | | 100 |
| Sage extract (Salvia off.) | | | | 75 | | | | | | | | |
| (5% Oleanolic/Ursolic acid) | | | | | | | | | | | | |
| Rosemary extract (90% Ursolic acid) | | 56 | | | | | | | 20 | | | |
| Boswellia serrata extract (40% Boswellic acid) | | | | | 150 | | 100 | | | | | |
| Eriobotrya japonica extract (1% corosolic acid) | | | | | | 48 | | | | | 36 | |
| Banaba extract (18% corosolic acid) | 6 | | 6 | 9 | | | | | 2.7 | | | |
| Banaba extract (2% corosolic acid) | | 24 | | | | | | | | 40 | | |
| Betula alba extract (10% Betulinic acid) | | 14 | | | | | | | | | | |
| Alpha-Lipoic acid | 100 | 100 | 10 | 75 | 115 | 100 | 75 | 80 | 120 | 75 | 100 | 100 |
| MCT oil (capric/caprylic triglycerides) | 120 | 160 | 280 | 320 | 320 | 160 | 400 | 340 | 180 | | 240 | |
| Capric/caprylic mono and diglycerides | 240 | 200 | | | | 150 | | | | | | 190 |
| (Capmul MCM) | | | | | | | | | | | | |
| Acetylated mono- and diglycerides | | | 100 | | | | | | 120 | | | |
| Linoleoyl polyoxyl-6 glycerides | | | 75 | | | 50 | | | | | | 90 |
| (Labrafil M2125CS) | | | | | | | | | | | | |
| D-Alpha-Tocopherol acetate | 80 | 60 | 30 | 30 | 40 | 50 | | | 80 | | | 58 |
| D-1-alpha Tocopherol | | | | | | 20 | | 60 | | 40 | 44 | 22 |
| Eugenol | | | 5 | | | 10 | | | | | | |
| Lemon oil | | | | | 8 | | | | | | | |
| Gaultheria procumbens essential oil | | | | | | | | 20 | | | | |
| Ubiquinol | | | | | | 30 | | | 50 | | | |
| Spearmint essential oil | 20 | | 10 | | | | | 8 | | | | |
| Cumin essential oil | | | 14 | | | | | | | | | |
| Lecithin | 45 | 45 | 35 | 35 | 55 | 50 | 45 | 30 | 50 | 40 | 40 | 40 |
| Polysorbate 60 | | | | | | 90 | | 100 | | | | 85 |
| Polysorbate 20 | 90 | 110 | | | | | | | | | | |
| Polysorbate 80 | | | | | | 80 | 85 | | | | 110 | |
| Hydrogenated polyoxyl castor oil | | | 90 | 115 | 60 | 60 | | 85 | | | | |
| Polyethoxylated castor oil | 110 | | | | | | | | | | | 80 |
| Sucrose stearate (Cisterna F-110) | | | | | | 100 | | 60 | | | | 90 |
| Sorbitan monooleate (Span 80) | | 35 | 60 | | | | 40 | | | | | 85 |
| Tocophersolan (TPGS) | | | | | | | | | 50 | 80 | 60 | |
| Total weight, mg | 861 | 804 | 757 | 659 | 748 | 998 | 745 | 923 | 753 | 325 | 630 | 990 |

TABLE 4
Comparative decrease of Fasting Plasma Glucose level after oral
administration of different formulations (rice bran filled soft gelatin
capsule and SEDDS filled capsule, respectively)

| | Banaba Extract | Corosolic | | | |
|---|---|---|---|---|---|
| | content (18% | acid dose, | | | |
| | Corosolic acid), | mg per | 1 | 2 | 1 |
| FORMULATIONS | mg | day | week | weeks | month |
| GlucoHelp ™ Banaba | 56.0 | 10.0 | 10% | 12% | ND |
| extract (18% CA) in a | | | | | |
| rice bran oil filled soft | | | | | |
| gel capsule * | | | | | |
| Self-nanoemulsifying | 12.0 | 2.16 | 12% | 18% | 25% |
| composition of Banaba | | | | | |
| extract (18% CA) and | | | | | |
| alpha-lipoic acid in a | | | | | |
| soft gel capsule (2 caps) | | | | | |

* Published data: [“GlucoHelp ™ - Banaba leaf extract standardized to 18% corosolic acid”. Brochure, Soft Gel technologies Inc., Los Angeles, CA]

TABLE 5
Droplet size and size distribution after emulsifying
with warm (37° C.) water containing media

| Amount of the acid per | Z- | | Particle size distribution | | | |
|---|---|---|---|---|---|---|
| capsule with SEDDS, | Average | | (PSD) by volume | | | |
| containing Banaba extract | diame- | | Peak | | Peak | |
| (18% Corosolic aid) | ter, nm | PDI | 1 | % | 2 | % |
| No ALA (Banaba | 432 | 0.829 | 146 | 30.4 | 689 | 69.6 |
| extract only) | | | | | | |
| 50 mg rac-ALA | 276 | 0.348 | 179 | 76.9 | 478 | 23.1 |
| 75 mg rac-ALA | 172 | 0.270 | 83.6 | 36.5 | 307 | 63.5 |
| 100 mg rac-ALA | 111 | 0.115 | 105 | 100 | | |
| 100 mg Caprylic acid | 232 | 0.446 | 180 | 27.8 | 841 | 72.2 |
| 100 mg Oleic acid | 373 | 0.531 | 145 | 3.1 | 2030 | 96.9 |

A small amount of the self-emulsifying composition was diluted with appropriate amount of the warmed water phase and was shaken for 30-45 seconds. The formed emulsion was filtered through syringe filter with 5 mcm membrane to remove insoluble matter, presented in the natural extract, and droplet size was measured using dynamic light scattering system ZetaSizer Nano-S (Malvern Co., UK).

It was clearly visible that the addition of ALA decreased droplet size of the formed emulsions loaded with PTA-containing herbal extract. Droplet size depends on the amount of added alpha-Lipoic acid. At 100 mg of alpha-Lipoic acid, the emulsion had a single population of oil droplets with a narrow and uniform distribution (PDI<0.2). At the same time, other fatty acids such as Caprylic acid or Oleic acid, demonstrated relatively slight influence on the formation of emulsions with smaller particle size and narrow particle size distribution, Table 5.

Table 4 presents comparative data of two different formulation containing Banaba leaf extract (GlucoHelpTm), standardized to 18% Corosolic acid. The first softgel composition contains 56 mg of the Banaba extract, suspended in a rice bran oil. The glucose lowering activity of this product provided by Soft Gel Technologies (one capsule a day, equivalent of 10.0 mg of Corosolic acid). The second composition, based on proposed SEDDS formulation, contains 12 mg of the Banaba extract. Being administered orally (2 capsules a day, equivalent to 2.16 mg of Corosolic acid), SEDDS formulation demonstrates more pronounced glucose lowering activity, exceeding that of oil filled formulation despite much lower content of the Banaba leaf extract.

All references and patents cited herein are incorporated herein by reference in their entirety.

### REFERENCES

1. M. Broniatowski et al., “Interactions of pentacyclic triterpene acids with cardiolipins and related phosphatidylglycerols in model systems” Biochimica et Biophysica Acta 1838 (2014) 2530-2538
2. U.S. Pat. No. 7,713,546 Udell et al. “Corosolic acid formulation and its application for weight-loss management and blood sugar balance” Filed Apr. 3, 2001 Issued May 11, 2010
3. United States Patent Application 20080038335 “Method, formulation, and use thereof for improved oral absorption of pharmaceuticals or nutrients” Filed Oct. 19, 2077
4. WIPO Patent Application WO/2005/037250 “Self emulsifying drug delivery systems for hydrophobic therapeutic compounds”
5. U.S. Pat. No. 6,656,970 B2 Burman et al. “Method and compositions for solubilization of pentacyclic triterpenes” Dec. 2, 2003
6. Xi, J. et al., “Formulation Development and Bioavailability Evaluation of a Self-Nanoemulsified Drug Delivery System of Oleanolic Acid” AAPS Pharm. Sci. Tech, (2009) Vol. 10, No. 1, pp. 172-182.
7. Tarr et al., “Enhanced intestinal absorption of Cyclosporine in rats through the reduction of emulsion droplet size” Pharm. Res. (1989) 6 (1) pp. 40-43.
8. De Shmidt et al., “Intestinal absorption of penclomedine from lipid vehicles in the conscious rat: contribution of emulsification versus digestibility” International Journal of Pharmaceutics 270 (2004) 109-118
9. Kang, B et al., “Development of self-microemulsifying drug delivery systems (SMEDDS) for oral bioavailability enhancement of simvastatin in beagle dogs” International Journal of Pharmaceutics 274 (2004) 65-73
10. Mueller E. et al., “Influence of a fat-rich meal on the pharmacokinetics of a new oral formulation of cyclosporine in a crossover comparison with the market formulation” Pharm. Res. (1994) 11 (1) pp. 151-5; “Improved dose linearity of cyclosporine pharmacokinetics from a microemulsion formulation. Pharm. Res. (1994) 11(2):301-4.
11. Myers R. et al., “Systemic bioavailability of penclomedine (NSC-338720) from oil-in-water emulsions administered intraduodenally to rats” International Journal of Pharmaceutics, 78 (1992) pp. 217-226
12. Buyukorturk F., et al., “Impact of emulsion-based drug delivery systems on intestinal permeability and drug release kinetics”. Journal of Controlled Release 142 (2010) pp. 22-30
13. GlucoHelp™—Banaba leaf extract standardized to 18% corosolic acid.
   Brochure, Soft Gel technologies Inc. 6982 Bandini Blvd., Los Angeles, Calif. 90040. Link: http://www.soft-gel.com/doc/SGTI_GlucoHelp-Brocure.pdf

What is claimed is:

1. A self-nanoemulsifying composition for enhanced oral absorption of poorly soluble triterpene acids, comprising:
a. at least one substantially pure triterpene acid or at least one plant extract containing said triterpene acid in amount from about 0.01% to about 80% by weight of the composition;
b. at least one physiologically acceptable aromatic solubilizer in amount from about 0.5% to about 80% by weight of the composition;
c. at least one physiologically acceptable surfactant or mixture of surfactants in amount from about 0.5% to about 80% by weight of the composition; and
d. at least one natural or synthetic phospholipid in amount from about 0.1% to about 50% by weight of the composition.

2. The self-nanoemulsifying composition of claim 1 wherein the composition is administered orally, sublingually or via a transmucosal route.

3. The self-nanoemulsifying composition of claim 1 wherein triterpene acid is a pentacyclic triterpene acid.

4. The pentacyclic triterpene acid of claim 3 wherein the pentacyclic triterpene acid is selected from the group consisting of Corosolic acid, Ursolic acid, Maslinic acid, Moronic acid, Oleanolic acid, Glycyrrhizic acid, Betulinic acid, Pomolic acid, Tormentic acid, Hyptadienic acid, Augustic acid, Uncaric acid, Boswellic acid, analogs thereof, salts thereof, and derivatives thereof.

5. The self-nanoemulsifying composition of claim 1, wherein said composition forms a submicron emulsion, nanoemulsion, microemulsion or micellar solution upon contact with water media or body fluid, and average size of oil droplets of the formed emulsion is selected from the group consisting of less than 500 nm, less than 300 nm, less than 200 nm, and less than 150 nm.

6. The self-nanoemulsifying composition of claim 1 wherein a triterpene acid is substantially dissolved.

7. The self-nanoemulsifying composition of claim 1, wherein said physiologically acceptable aromatic solubilizer is selected from the group of chromones, chromanes, tocols, tocopherols and tocopherol esters, tocotrienoles, benzoic acid esters, hydroxybenzoic acid esters, eugenol, anisole, anethole, flavones, isoflavones, flavonoids, indoles, quinones, ubidecarenone, curcuminoids, derivatives thereof, and mixtures thereof.

8. The self-nanoemulsifying composition of claim 1 wherein said composition is administered as a solid dosage form.

9. The self-nanoemulsifying composition of claim 8 wherein said solid dosage form is a compressed tablet to be swallowed.

10. The self-nanoemulsifying composition of claim 8 wherein said solid dosage form is a compressed chewable, sublingual or buccal tablet.

11. The self-nanoemulsifying composition of claim 8 wherein said solid dosage form is a lozenge or a capsule.

12. The self-nanoemulsifying composition of claim 11 wherein said capsule is a liquid filled hard shell capsule or liquid filled soft shell capsule.

13. The self-nanoemulsifying composition of claim 11 wherein said capsule is filled with a self-nanoemulsifying composition.

14. The self-nanoemulsifying composition of claim 1 wherein the composition is administered as a liquid dosage form.

15. The self-nanoemulsifying composition of claim 14 wherein said liquid dosage form is administered in a diluted form or in a non-diluted form.

16. A self-nanoemulsifying composition administered via oral or intraoral routes,
wherein the composition spontaneously forms a submicron oil-in-water emulsion when contacted with water containing media and wherein the composition comprises:
a. a substantially pure pentacyclic triterpene acid or plant extract containing at least one pentacyclic triterpene acid in an amount of about 0.01 to 80% by weight of the composition;
b. a physiologically acceptable hydrophobic material forming an oil phase of the emulsion on contact with water containing media or body fluid in an amount of about 5% to about 60% by weight of the composition, wherein the pentacyclic triterpene acid remains substantially dissolved in the oil phase of the emulsion;
c. a physiologically acceptable surfactant or mixture of surfactants in an amount of about 0.5% to about 80% by weight of the composition;
d. at least one natural or synthetic phospholipid in amount from about 0.1% to about 50% by weight of the composition; and
e. D-, L-, racemic alpha-lipoic acid, mixture thereof or physiologically acceptable salts thereof in amount from about 1 to about 50% by weight of the composition.

17. A self-nanoemulsifying composition for enhanced oral absorption of poorly soluble triterpene acids, comprising:
a. at least one pure triterpene acid or at least one plant extract containing said triterpene acid in amount from about 0.01% to about 80% by weight of the composition;
b. at least one physiologically acceptable aromatic solubilizer in amount from about 0.5% to about 80% by weight of the composition;
c. at least one physiologically acceptable surfactant or mixture of surfactants in amount from about 0.5% to about 80% by weight of the composition;
d. at least one natural or synthetic phospholipid in amount from about 0.1% to about 50% by weight of the composition; and
e. R-alpha-lipoic acid, S-alpha-lipoic acid, racemic alpha-lipoic acid or a physiologically acceptable salt thereof in amount from about 0.5% to about 50% by weight of the composition.

18. The self-nanoemulsifying composition of claim 17 wherein the composition is administered orally, sublingually or via a transmucosal route.

19. The pentacyclic triterpene acid of claim 16 wherein the pentacyclic triterpene acid is selected from the group consisting of Corosolic acid, Ursolic acid, Maslinic acid, Moronic acid, Oleanolic acid, Glycyrrhizic acid, Betulinic acid, Pomolic acid, Tormentic acid, Hyptadienic acid, Augustic acid, Uncaric acid, Boswellic acid, analogs thereof, salts thereof, and derivatives thereof.

20. The pentacyclic triterpene acid of claim 16 wherein the pentacyclic triterpene acid is selected from the group consisting of Corosolic acid, Ursolic acid, Maslinic acid, Moronic acid, Oleanolic acid, Glycyrrhizic acid, Betulinic acid, Pomolic acid, Tormentic acid, Hyptadienic acid, Augustic acid, Uncaric acid, Boswellic acid, analogs thereof, salts thereof, and derivatives thereof.
